(19) United States
(12) Patent Application Publication
(10) Pub. No.: US 2013/0143197 A1
(43) Pub. Date: Jun. 6, 2013

(54) Microfluidic Cell Separation in the Assay of Blood

(71) Applicant: Herbert Heyneker, San Francisco, CA (US)

(72) Inventor: Herbert Heyneker, San Francisco, CA (US)

(73) Assignee: GPB SCIENTIFIC, LLC, Richmond, VA (US)

(21) Appl. No.: 13/816,933

(22) PCT Filed: Aug. 12, 2011
(86) PCT No.: PCT/US11/47654

§ 371 Date: Feb. 13, 2013

Related U.S. Application Data
(60) Provisional application No. 61/373,866, filed on Aug. 15, 2010.
(60) Provisional application No. 61/383,710, filed on Sep. 16, 2010.

(51) Int. Cl. G01N 33/569

(52) U.S. Cl. CPC G01N 33/56972
USPC 435/2; 435/287.2

(57) ABSTRACT

The present invention is directed to methods for assaying blood samples to quantitate the types of white blood cells present. In addition, the invention includes equipment that can be used for these methods. One feature of the methodology is the use of micro fluidic devices for the separation of white blood cells from red blood cells.

### CROSS REFERENCE TO RELATED APPLICATIONS

The present application claims the benefit of U.S. provisional application 61/383,710, filed on Sep. 16, 2010 and U.S. provisional application 61/373,866, filed on Aug. 15, 2010 the contents of which are hereby incorporated by reference in their entirety.

### FIELD OF THE INVENTION

The present invention is directed to procedures that can be used to rapidly analyze the different types of cells present in blood samples and to a system that can be used in carrying out these procedures.

### BACKGROUND OF THE INVENTION

Human blood is comprised of three main types of cells: red blood cells (RBCs), white blood cells (WBCs) and platelets. WBCs occur in several structurally and functionally distinct forms and may be classified as neutrophils, eosinophils, basophils, lymphocytes, monocytes and macrophages. Abnormal levels of these cells are associated with a number of serious diseases such as leukemia, agranulocytosis, and AIDS. Thus, the ability to detect abnormalities in the levels of specific types of WBCS is of considerable diagnostic interest. This is particularly true with respect to AIDS, in which patients have CD4⁺ T lymphocyte levels much lower than the levels in normal individuals.

RBCs are generally smaller than WBCs but are present in much larger quantities (see US 2007/0160503). It is therefore generally advantageous to remove RBCs prior to attempting to quantitate levels of WBC types. Although a crude separation of RBCs and WBCs can be achieved by centrifugation, this method is ineffective at distinguishing the types of WBCs present. More specific procedures such as flow cytometry and cell sorting procedures (Bauer, J. Chromatog. B, 722:55-69 (1999); Anderson, et al., Proc. Natl. Acad. Sci. USA 93:8508-8511 (1996); Moore, et al., J. Biochem. Biophys. Methods 37:1-2 (1998)) may be used but the preparation of samples for these procedures may not lend itself to automation and may involve the lysis of cells and release of materials that have the potential of interfering with analyses.

### SUMMARY OF THE INVENTION

The present invention is based upon the adaptation of microfluidic separations (particularly size separations) to the analysis of blood samples for levels of different types of cells. The method is of value for the rapid testing of samples for white blood cell levels suggestive of the presence of cancer or AIDS. The methodology may also be used to monitor AIDS patients to determine whether the disease is progressing. Because the technology is simple to use and lends itself to automation, it should be of value in clinical chemistry laboratories and in screening procedures.

Systems for Performing Assays

In its first aspect, the invention is directed to a system for assaying the types of cells present in a sample of blood. The system includes: a) a reaction chamber; b) a microfluidic device capable of separating red blood cells from white blood cells; c) a pump in fluid connection with the device which is capable of impelling the flow of fluid through the device; d) an analyzer that is in fluid connection with an outlet of the device and which is capable of performing an optical or chemical analysis of materials that have been separated; and e) a data output device that may either be part of the analyzer or separate from it.

The reaction chamber (component “a” above) is in fluid connection with the microfluidic device and must have at least one opening or port allowing the introduction of samples (typically blood samples) and reagents (typically detectably labeled antibodies that bind preferentially to a particular type of WBC). The term “fluid connection” as used herein means that there must be a pathway allowing the flow of fluid from one part of the system, e.g., the reaction chamber, to another part of the system, e.g., the microfluidic device. Typically the pathway will be provided by plastic or metal tubing.

The microfluidic device must be capable of separating white blood cells from red blood cells and a description of devices appropriate for this purpose is provided more fully below. The device must have at least one inlet port or opening which is in fluid connection with at least one outlet port or opening of the reaction chamber and which, during operation, receives blood samples from the reaction chamber. There must also be at least one outlet port usually located on the opposite side of the device, through which material may exit. Preferably there are at least two outlet ports, one of which is positioned to convey fluid which, relative to whole blood samples, is enriched in WBCs and one of which is positioned to convey fluid containing RBCs and platelets but relatively few WBCs. These ports or openings may optionally include valves that can be opened or closed by someone operating the device.

Generally, the reaction chamber and the microfluidic device will be separate from one another, but in an alternative design, the reaction chamber may be integrated into the device itself. There must be at least one microfluidic channel running from the inlet port or opening of the device to its outlet port or opening and it is within this channel, or these channels, that the separation of materials takes place. The term “microfluidic channel” as used herein refers to a pathway for fluid having at least one cross-sectional dimension in the range of 10 nm to 1 mm.

The most preferred microfluidic devices are those that separate cells and other materials based upon their size. Sizing devices may accomplish separations by means of an array of obstacles, posts or barriers that create a network of gaps within microfluidic channels. When fluid flows through the channel, it is divided unequally into a major flux component and a minor flux component as it passes through the network of gaps. This results in the average direction of the major flux component being nonparallel to the average direction of the flow field. The obstacles within the microfluidic channel should be arranged such that, when blood cells are passed through the device, white blood cells are transported generally in the average direction of one flux component and red blood cells are transported generally in the average direction of the other flux component, thereby separating the cells according to size.

The assay system must include a means for generating a force that impels the materials to be separated through the device. Any way to generate such a force that has been described in the art may be used for this purpose. Thus, the system may use devices that generate electrical, electrophoretic, electro-osmotic, centrifugal, gravitational, hydrodynamic, pressure gradient, or capillary forces. Most preferably, one or more pumps will be used to create a hydrodynamic force that propels fluid flow. The pump(s) must be in fluid connection with either an inlet or outlet port on the device and must generate sufficient force to propel fluid through the microfluidic channel(s). A pump may be connected directly to a port or opening on the device or it may be connected indirectly. For example, the pump may be connected to the reaction chamber and create a force that is transmitted by fluid connection from the reaction chamber to the device.

At least one outlet port or opening on the microfluidic device (in particular a port or opening positioned to convey fluid which is enriched in WBCs) must be in fluid connection with an inlet port or opening on the analyzer that allows the analyzer to receive materials that have been separated by the device for optical or chemical analysis. Examples of types of analyzers that may be used include flow cytometers, spectrophotometers, fluorescence detectors and radioactivity counters. The most preferred of these is a fluorescence detector.

Typically, the analyzer will be separate from the microfluidic device but it is also possible for the analyzer to be integrated as part of the device itself. Preferably, the microfluidic device has at least one port or opening that leads to an analyzer and a second port or opening that leads to a collection vessel used for collecting RBCs, platelets and other materials smaller than WBCs.

Finally, the system for analyzing types of cells must have a data output device for printing or displaying results from the analyzer. Usually this will be a computer or printer that displays the results of an optical or chemical analysis. The data output device may either be separate from, or part of, the analyzer.

Assay Procedures

In another aspect, the invention is directed to a method of assaying a blood sample to determine the amount of different cell types present. The method involves first obtaining a test blood sample and incubating it with one or more detectably labeled antibodies that: a) do not bind to red blood cells to a substantial degree, and b) bind preferentially to one or more target white blood cells. The phrase “do not bind to red blood cells to a substantial degree” as used herein means that an antibody has at least a 1000 fold lesser affinity for red blood cells than for a target white blood cell, i.e., the WBC it was designed to detect. Preferably, the affinity is at least 10,000 or 100,000 fold less. The phrase “bind preferentially to one or more target white blood cells” as used herein means that the antibody has at least a 100-fold greater affinity for one particular type of white blood cell than for any other type. For example, if the antibody was designed to recognized CD4⁺ T lymphocytes, it would bind to these cells with at least a 100 fold greater affinity than to any other lymphocytes or white blood cells. A difference of greater than 1000 or 10,000 is preferred. The phrase “detectably labeled antibodies” as used herein means that the antibodies are attached to a molecule or compound that can be detected using standard laboratory techniques. For example, the antibodies may be attached to a radioactive isotope such as ¹²⁵I or to a fluorescent tag such as fluoresceine isothiocyanate (FITC). The most preferred detectable label is phycoerythrin (PE). The incubation between blood and detectably labeled antibody is carried out under conditions, and for a period of time, sufficient to allow the formation of antibody-cell complexes.

The complexes formed are next separated from red blood cells and from unbound antibody using a microfluidic device. In a preferred embodiment, the complexes are pumped from a reaction chamber through a device that separates cells based on size. This will typically result in white blood cells exiting the device at different location than red blood cells and unbound label.

The separated white blood cells are collected and analyzed to determine the amount of detectable label present. Depending on the type of label used, analysis may be performed with a flow cytometer, spectrophotometer, radioactivity counter, fluoresence detector or other equipment. In automated analyses carried out using a system such as that described above, cells would be routed from the outlet of the microfluidic device and directly into the analyzer, i.e., there would not be a separate collection step. For example, fluorescently labeled complexes may be pumped into a flow cytometer. The results from the analyzer will typically be recorded on a data output device, i.e. a device that prints or displays the results. Often this will be a computer or printer that is incorporated into the analyzer. However, the data output device may also be separate from the analyzer.

Typically, the results obtained from a test sample will be compared to results obtained from one or more control samples. The control samples may be, for example, derived from healthy people and will provide a “normal” range of white blood cell levels. A comparison between test and control samples will reveal whether a group of T cells is abnormally elevated or depleted and may suggest the presence of disease. Although knowledge of a normal range of cells is of diagnostic value, it is not absolutely necessary to run a separate control sample for each assay in order to make comparisons. For example, one control may be used for multiple assays or a comparison can simply be made between test results and a known normal range.

In an especially preferred embodiment, the assay method uses antibodies that preferentially bind to lymphocytes (preferably CD4⁺ T lymphocytes) to help determine if a person has AIDS. In general, CD4⁺ T lymphocyte levels of 200 cells per mm³ or less would be an indication that AIDS is present, whereas levels of roughly 500-1600 cells per mm³ would be considered normal. In cases where a patient has already been diagnosed as having AIDS, periodic assays may be run to determine whether CD4⁺ levels are changing and therefore whether the disease appears to be progressing or responding to therapy.

Alternatively, antibodies that preferentially bind to CD8⁺ T cells may be used and the method may serve as a diagnostic test for cancer. For example, abnormally elevated levels of these cells may indicate the presence of an adenocarcinoma; melanoma; myeloma; sarcoma; teratocarcinoma and especially a leukemia or lymphoma. Particular organs affected may include the adrenal gland, bladder, bone, bone marrow, brain, breast, cervix, gall bladder, colon, stomach, heart, kidney, liver, lung, muscle, pancreas, parathyroid, prostate, thyroid or uterus.

The assays should also be useful for other diseases in which levels of specific leukocytes or classes of leukocytes would be expected to change. This would include inflammatory diseases or conditions which, for the purposes of the present invention, include atherosclerosis, asthma, autoimmune diseases (e.g., lupus or multiple sclerosis), inflammatory bowel diseases (e.g., Crohn's disease or ulcerative colitis), rheumatoid arthritis, various allergies and transplant rejection. For example a change in macrophage or granulocyte levels (e.g., an increase in the number of these cells in the blood of test subjects compared to control samples from healthy individuals or the population as a whole) may suggest the presence of disease.

The method can also be used to compare the levels of two or more different types of white blood cells which may be of value diagnostically or to researchers examining the effects of diseases and disease treatments. A comparison can be obtained, for example, by using two or more antibodies that bind preferentially to different target cells and that have distinct labels. The term “distinct labels” as used herein means that the analyzer, or analyzers, being used in the method can distinguish the labels when they are together. The method can be used to compare the levels of two different types of cells (e.g., neutrophils, basophils, eosinophils, monocytes, macrophages and dendritic cells) or to compare cells with more specific characteristics within a single type (e.g., CD4⁺ T cells and CD8⁺ T cells). In addition, fluorescence-activated cell sorting may be used to further separate cell types obtained from a microfluidic device so that further testing can take place.

The use of multiple distinctly labeled antibodies as described above would allow a ratio to be determined between different classes of leukocytes, e.g., leukocytes expected to change in response to the presence of disease and leukocytes that would not be expected to change. This would help to control for changes in specific leukocyte levels due to assay variability. Of particular interest in this regard is the use of antibodies specific for CD45 to broadly measure leukocyte levels together with an antibody specific for one particular leukocyte type, e.g., an antibody specific for CD4⁺ T cells or CD8⁺ T cells. For example, an assay useful in helping to identify patients with AIDS might use an antibody against CD45 labeled with a Cy3 fluorescent dye together with an antibody specific for CD4 labeled with Cy5 fluorescent dye. The ratio of CD4/CD45 cells (or CY5/CY3 label) may then be used to assess a blood sample, with abnormally low values suggesting the presence of AIDS.

The assay method can be automated using a system having the components described herein, i.e., a) a reaction chamber; b) a microfluidic device capable of separating red blood cells from white blood cells; c) a pump in fluid connection with the device which is capable of impelling the flow of fluid through the device; d) an analyzer that is in fluid connection with an outlet of the device and which is capable of performing an optical or chemical analysis of materials that have been separated; and e) a data output device that may either be part of the analyzer or separate from it. The system may also include a buffer reservoir that is separate from the reaction chamber, microfluidic device, analyzer and data output device.

### BRIEF DESCRIPTION OF THE FIGURES

FIG. 1 is a schematic drawing showing various components of an assay system. The portions with diagonal lines are ports or openings leading into or out of a component, each of which may optionally include a valve. “A” in the FIGURE represents a reaction chamber where a blood sample and labeled antibodies may be combined. If desired, the reaction chamber can be heated and/or agitated to promote mixing. The reaction product, typically including antibody/antigen complexes, is pumped from the reaction chamber into a microfluidic device (D) that is capable of separating red blood cells from white blood cells (preferably based on size). The FIGURE shows the reaction chamber and the microfluidic device as separate components by it is also possible to integrate the reaction chamber into the microfluidic device. “B” represents pumps that, in the drawing, are connected to the reaction chamber (A) and to a buffer reservoir (C), These pumps provide a force for propelling material through the system. Once on the microfluidic device, white blood cells (WBCS) are diverted in the direction of an outlet leading into an analyzer (E) where the amount of bound label is determined. “F” is a data output device (depicted in the FIGURE as a computer monitor) that presents the results from the analyzer. The data output device may either be part of the analyzer or separate from it. Platelets, red blood cells (RBCS) and other materials are directed to a separate collection vessel (G).

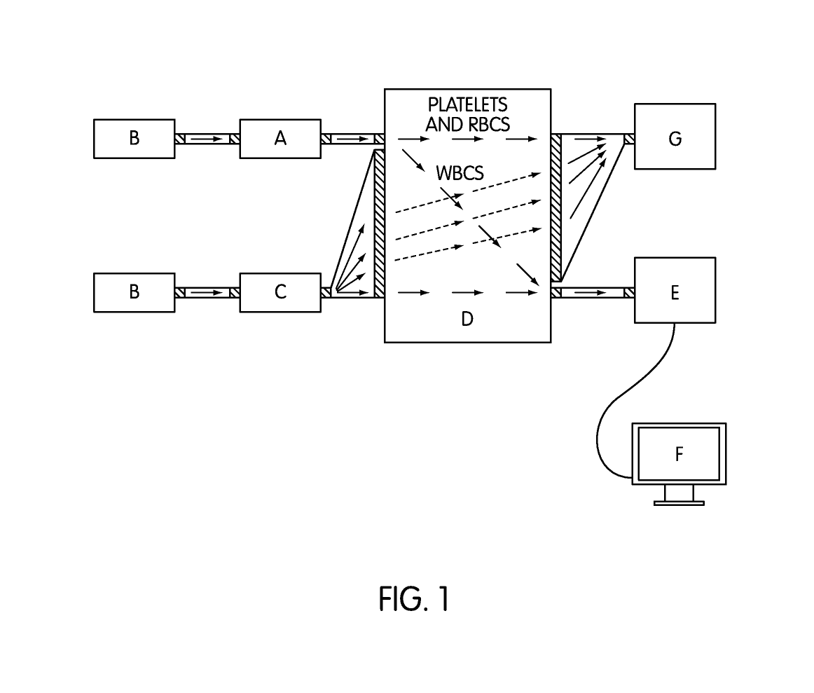

### DETAILED DESCRIPTION OF THE INVENTION

The present invention is directed to a system for assaying cells in blood samples and to an assay procedure that uses microfluidic devices to carry out cell separations. Apart from the arrangement of components and the use of microfluidic devices, the reaction chambers, pumps and analyzers that make up the assay system are standard in the art of clinical chemistry and can be purchased commercially from multiple manufacturers.

Assay Protocol

The assay for determining the amounts of different cell types within a blood sample will vary somewhat depending on specific objectives. However, its essential features are as follows.

The initial step involves the collection of blood, typically in the presence of an anticoagulant such as EDTA, heparin, citrate, etc. The anticoagulated blood is mixed with a factor that binds specifically to one or more (typically one) type of white blood cell. Examples of binding factors that may be used include proteins, aptemers, synthetic molecules and, most preferably, antibodies that bind to surface markers on the cells of interest (e.g., CD4, CD3, CD8, CD14, CD19, surface proteins, carbohydrates, lipids, etc). The binding factor must be detectably labeled, i.e., it either must naturally have, or be modified to have, a feature that allows it to be quantitatively assayed. Examples of labels that may be attached for this purpose include fluorescent labels, colored labels, magnetic labels, and radioactive labels.

After mixing, the blood sample and the binding factors are incubated under conditions, and for a period of time, sufficient to allow the formation of complexes between the detectably labeled binding factors and the cells that they specifically recognize. Multiple cell types can be assessed from in a single assay by using differently labeled binding factors and detection systems that can distinguish between the labels.

Once complexes have been formed, white blood cells (including those attached to a binding factor) are separated from red blood cells, platelets, plasma, and unbound label using a microfluidic device (preferably a device that separates cells on the basis of size). Passing the cells through the device also has the effect of transferring them into a physiological buffer such as phosphate buffered saline, Hank's balanced salt solution.

Finally, the recovered white blood cells are assayed to determine the quantity of labeled binder that they contain. Because the unbound labeled molecules and interfering red blood cells, plasma, platelets, etc. are largely removed from the white blood cells, the quantity of labeled binder attached to the white blood cells will be directly related to the amount of the cells of interest in the sample.

Although not preferred, it is possible to store separated white blood cells prior to performing an analysis of the amount of label present. Since detection does not require viable or intact cells after separation, sample storage is dependent on the stability of labeled binder used.

One advantage of the assay method is that it lends itself readily to automation and to the handling of large numbers of blood samples. Since a primary feature of AIDS is a deficiency in CD4⁺ T lymphocytes, the method is especially well suited to the detection or monitoring of this disease. Other advantages are that small samples of blood (e.g., 0.5 or less) may be assayed, the method allows for the rapid removal subtances that may interfere with assays and that separations based on size are relatively gentle allowing for the potential recovery of intact cells for further study.

Microfluidic Devices

Any of the microfluidic devices that have been described in the art that are capable of separating red blood cells and white blood cells may be used for the present invention. Especially preferred are devices that are capable of carrying out separations based on size. Such devices include those described in U.S. Pat. No. 5,837,115; U.S. Pat. No. 7,150,812; U.S. Pat. No. 6,685,841; U.S. Pat. No. 7,318,902;7,472,794; and U.S. Pat. No. 7,735,652; all of which are hereby incorporated by reference in their entirety. Other references that provide guidance that may be helpful in the making and use of devices for the present invention include: U.S. Pat. No. 5,427,663; U.S. Pat. No. 7,276,170; U.S. Pat. No. 6,913,697; US 2006/0134599; US 2007/0160503; US 20050282293; US 2006/0121624; US 2005/0266433; US 2007/0026381; US 2007/0026414; US 2007/0026417; US 2007/0026415; US 2007/0026413; US 2007/0099207; US 2007/0196820; US 2007/0059680; US 2007/0059718; US 2007/005916; US 2007/0059774; US 2007/0059781; US 2007/0059719; US 2006/0223178; US 2008/0124721; US 2008/0090239; and US 2008/0113358; all of which are also incorporated by reference herein in their entirety.

Of the various references describing the making and use of devices, U.S. Pat. No. 7,150,812 provides particularly good guidance and U.S. Pat. No. 7,735,652 is of particular interest in that it is particularly concerned with microfluidic devices for separations performed on blood samples (in this regard, see also US 2007/0160503) and describes ways to prevent the clogging of devices (preferably also used in the devices employed in the methods disclosed herein).

The '812 patent describes a preferred device in which there is a channel with an ordered array of obstacles arranged asymmetrically with respect to the direction of a force field applied to propel fluid through the device. The obstacles form a network of gaps that, in the presence of fluid flow, create a field pattern such that the field flux from a gap is divided unequally into a major flux component and a minor flux component. Particles passing through the device of a similar size will usually be diverted in the same direction, i.e., diverted to the same side of an obstacle, whereas particles of a different size may be diverted in a different direction. Therefore, it is possible to form an array of obstacles that takes advantage of differences in the size of RBCs and WBCs to effect their separation.

According to U.S. Pat. No. 7,735,652, U.S. Pat. No. 7,150,812 and Huang, et al., Science 304:987-990 (2004) disclose the basic separation principles of deterministic lateral displacement, a process referred to in '652 as “bumping.” Displacement may be accomplished in an array in which each row of obstacles has a row shift fraction of one third, which creates three equal flux streamlines. Small particles stay within a flow stream and large particles are displaced at each obstacle. Theoretical considerations with respect to separations in such devices are discussed in detail in '652. In addition, this reference describes the removal of large, separated objects by providing an alternate pathway to prevent clogging or jamming downstream.

### Examples

The current prophetic example is meant to illustrate how a blood sample could be assayed to determine if it is obtained from a patient with AIDS.

In a first step, blood is collected from a patient in an EDTA containing vacutainer tube (˜1.8 mg EDTA per ml blood). 50 ul of anti-coagulated blood is mixed with 20 ul of phycoerythrin (PE) labeled anti-CD4 antibody. The mixture is incubated for 15 minutes at room temperature in the dark and is then mixed with 70 ul of degassed phosphate buffered saline (without calcium and magnesium, and containing 1% bovine serum albumin and 2 mM EDTA). A 140 ul blood cell/antibody/buffer aliquot is applied to a microfluidic separation device designed to separate blood cells by size. The sample is propelled through the device using the degassed phosphate buffered saline as the running buffer. As the sample runs through the device, white blood cells are moved into the running buffer stream and the remaining blood components and unbound labeled binder continue through the chip into the waste stream. The collected white blood cell fraction is then assayed for PE by fluorescence activation and detection. The fluorescence levels directly reflect the amount of CD4⁺ T cells present.

All references cited herein are fully incorporated by reference. Having now fully described the invention, it will be understood by those of skill in the art that the invention may be practiced within a wide and equivalent range of conditions, parameters and the like, without affecting the spirit or scope of the invention or any embodiment thereof.

1. A system for assaying the types of cells present in a sample of blood comprising:
a) a reaction chamber with at least one opening or port allowing the introduction of a blood sample and detectably labeled antibodies and at least one outlet opening or port through which medium from said reaction chamber can flow;
b) a microfluidic device comprising at least one inlet port or opening which is in fluid connection with said outlet port or opening from said reaction chamber and at least one outlet port or opening through which material exiting from said device may pass, wherein said device is capable of separating white blood cells from red blood cells and wherein said reaction chamber is either separate from or integrated into said microfluidic device;
c) at least one pump which is in connection with either an inlet or outlet port on said device in such a manner to allow the pump to provide a force sufficient to propel fluid from an inlet port or opening on said device to an outlet port or opening on said device;
d) an analyzer comprising an inlet port or opening in fluid connection with an outlet port or opening on said microfluidic device, wherein said analyzer is capable of performing an optical or chemical analysis of materials flowing from an outlet on said microfluidic device to an inlet on said analyzer;
e) a data output device for printing or displaying results from said analyzer.

2. The system of claim 1, wherein said reaction chamber is separate from said microfluidic device and said microfluidic device comprises at least two outlet ports or openings, wherein at least one outlet or opening is in fluid connection with said analyzer and at least port or opening is in fluid connection with a separate collection vessel.

3. The system of claim 2, wherein said analyzer is a flow cytometer, a spectrophotometer, fluorescence detector or a radioactivity counter.

4. The system of claim 2, wherein said analyzer is a flow cytometer and an outlet port or opening of said microfluidic device is located on the opposite side of the device relative to an inlet port or opening.

5. The system claim 2, further comprising a buffer reservoir, separate from said reaction chamber, microfluidic device, analyzer and data output device, wherein said buffer reservoir is in fluid connection with said microfluidic device.

6. The system of claim 1, wherein said microfluidic device separates samples based on size.

7. The system of claim 6, wherein said microfluidic device comprises a microfluidic channel having a network of gaps and, upon the flow of fluid through said microfluidic channel, a flux of said flow from the gaps is divided unequally into a major flux component and a minor flux component.

8. A method of assaying a blood sample to determine the amount of different cell types present comprising:
a) obtaining a test blood sample;
b) incubating said test blood sample with one or more detectably labeled antibodies, wherein:
i) said antibodies do not bind to red blood cells to a substantial degree
ii) said antibodies bind preferentially to one or more target white blood cells;
iii) said incubation results in the formation of antibody-cell complexes;
c) separating said antibody-cell complexes from said red blood cells and from unbound antibody using a microfluidic device;
d) quantitating the amount of detectable label in the separated antibody-cell complexes obtained in the separation of step c) to determine the amount of target white blood cell present.

9. The method of claim 8, further comprising comparing the results obtained in step d) with results from one or more control samples and concluding that said target white blood cells are abnormally high or low based upon this comparison.

10. The method of claim 9, wherein said antibodies preferentially bind to lymphocytes.

11. The method of claim 10, wherein separate antibodies from those preferentially binding to lymphocytes are incubated with said test blood sample, said separate antibodies preferentially binding to one or more target cells selected from the group consisting of: neutrophils, basophils, eosinophils, monocytes, macrophages and dendritic cells and wherein, said separate antibodies have a detectable label that is different from the detectable label on antibodies recognizing said lymphocytes.

12. The method of claim 10, wherein said lymphocytes are T lymphocytes.

13. The method of claim 11, wherein said lymphocytes are CD8⁺ T lymphocytes.

14. The method of claim 9, wherein said blood sample is obtained from an individual as part of a test to determine whether said individual has AIDS or from a patient known to have AIDS to determine whether the disease is progressing.

15. The method of claim 14, wherein said antibodies bind preferentially to CD4⁺ T lymphocytes.

16. The method of claim 15, wherein said antibodies are labeled with fluorescent label and are quantitated by flow cytometry.

17. The method of claim 8, further comprising a separation of cells by fluorescence-activated cell sorting.

18. The method of claim 8, wherein said microfluidic device separates cells based on size.

19. The method of claim 8, wherein said assay is carried out using the system of claim 1 and 0.25-0.5 ml of blood sample is used.

20. The method of claim 19, wherein said system further comprises a buffer reservoir, separate from said reaction chamber, microfluidic device, analyzer and data output device, wherein said reservoir is in fluid connection with said microfluidic device.
